(19) United States
(12) Patent Application Publication
(10) Pub. No.: US 2025/0072768 A1
(43) Pub. Date: Mar. 6, 2025

(54) WRIST-WEARABLE BLOOD PRESSURE MEASUREMENT DEVICE

(71) Applicant: CHARMCARE CO., LTD., Seoul (KR)

(72) Inventor: Dong Hwa LEE, Yongin-si (KR)

(21) Appl. No.: 18/518,331

(22) Filed: Nov. 22, 2023

Related U.S. Application Data
(63) Continuation of application No. PCT/KR2023/018024, filed on Nov. 10, 2023.

(30) Foreign Application Priority Data
Sep. 1, 2023 (KR) 10-2023-0115977

(51) Int. Cl. A61B 5/021; A61B 5/00; A61B 5/022

(52) U.S. Cl. CPC A61B 5/02141; A61B 5/022; A61B 5/681

(57) ABSTRACT

A wrist-wearable blood pressure measurement device includes a strap to wrap a wrist of a subject, a blood pressure measurement sensor placed to be adjacent to an artery of the subject, a blood pressure measurement module to receive data measured by the blood pressure measurement sensor, and an angle sensor to detect a rotation state of the wrist of the subject. The angle sensor measures an angle at which an inner side surface of the wrist rotates around a vertical reference axis perpendicular to a ground. The blood pressure measurement module determines that the angle is in a normal state when the angle is within an allowable range, and determines the angle is in an abnormal state when the angle is not within the allowable range, and determines at least one of whether to continue measurement, whether to provide guidance, and whether a measurement value is valid.

### BACKGROUND

The present disclosure relates to a wrist-wearable blood pressure measurement device.

Blood pressure information is one of the important pieces of information in determining a health state. Blood pressure refers to the pressure that blood exerts on the walls of blood vessels. When the heart contracts and pushes out blood, the pressure of the blood vessels is called a systolic blood pressure or a systolic blood pressure, and when the heart relaxes and accepts blood, the pressure of the blood vessels is called a diastolic blood pressure or a diastolic blood pressure.

In general, the systolic blood pressure of healthy people is less than 120 mmHg and the diastolic blood pressure thereof is less than 80 mmHg. In addition, people whose systolic blood pressure is 140 mmHg or more and diastolic blood pressure is 90 mmHg or more have a high blood pressure, and people whose systolic blood pressure is 90 mm Hg or less and diastolic blood pressure is 60 mm Hg or less have a low blood pressure.

When the blood pressure is high or low, abnormal health signals may be generated, and accordingly, it is important to measure blood pressure to obtain blood pressure information. Methods for measuring the blood pressure generally include an auscultation method, an oscillometric method, and a tonometric method.

Among the methods, the oscillometric method is a method of measuring systolic and diastolic blood pressures by detecting pulse waves generated in the process of sufficiently pressurizing a body part through which arterial blood passes and then decompressing at a constant speed to block arterial blood flow, or in the process of increasing and pressurizing the body part through which arterial blood passes at a constant speed.

The tonometric method is a method of applying a certain amount of pressure to a body part so as not to completely block blood flow in the artery and measuring blood pressure by using sizes and shapes of the pulse waves generated at this time.

Most known devices for measuring a subject's blood pressure by using the oscillometric method are configured to measure blood pressure by wrapping a device around the subject's arm. However, the known blood pressure measurement device designed to measure blood pressure by wrapping the device around a subject's arm has the problem of being bulky and inconvenient to carry.

In order to solve this problem, Korean Patent No. 10-1349767 (hereinafter referred to as “prior document”) discloses a wristwatch-type blood pressure monitor that is worn on a subject's wrist to improve portability and includes an air chamber capable of pressurizing the subject's arteries. The wristwatch-type blood pressure monitor of the prior document may measure the pressure of an air chamber when the air chamber pressurizes the subject's artery and is configured to calculate the subject's blood pressure based on the measured pressure of the air chamber.

However, when the wrist-wearable blood pressure monitor of the prior document measures blood pressure on the wrist under the palm, and when the wrist is bent inward, the skin in the area where blood pressure is measured is folded or thickened, causing errors in blood pressure measurement.

### SUMMARY

The present disclosure provides a wrist-wearable blood pressure measurement device that may accurately measure blood pressure by guiding the inside of a wrist wearing the wrist-wearable blood pressure measurement device to face a direction perpendicular to the ground or an opposite direction thereof while measuring blood pressure.

Objects to be solved by the present disclosure are not limited to the objects described above, and there may be other objects.

According to an aspect of the present disclosure, a wrist-wearable blood pressure measurement device includes a strap configured to wrap a wrist of a subject, a blood pressure measurement sensor placed to be adjacent to an artery of the subject, a blood pressure measurement module configured to receive data measured by the blood pressure measurement sensor, and an angle sensor configured to detect a rotation state of the wrist of the subject, wherein the angle sensor measures an angle at which an inner side surface of the wrist rotates around a vertical reference axis perpendicular to a ground, and the blood pressure measurement module determines that the angle is in a normal state when the angle measured by the angle sensor is within an allowable angle range, and determines the angle is in an abnormal state when the angle measured by the angle sensor is not within the allowable angle range, and determines at least one of whether to continue measurement, whether to provide guidance, and whether a measurement value is valid.

Also, the allowable angle range may be greater than or equal to 0 degrees and less than or equal to 90 degrees.

Also, when the blood pressure measurement module determines that the angle of the wrist of the subject is in an abnormal state, guidance may be provided to the subject to adjust the inner side surface of the wrist to be parallel to the vertical reference axis or the inner side surface of the wrist to be perpendicular to the vertical reference axis to face sky.

Also, when the blood pressure measurement module determines that the angle of the wrist of the subject is in a normal state, notification information may be output to inform the subject that the angle is in the normal state.

Also, the wrist-wearable blood pressure measurement device may output notification information indicating that the rotation state of the wrist is in the normal state or notification information indicating that the rotation state of the wrist is in the abnormal state through vibration notification, voice notification, a display screen output, or an air pressure of a cuff.

Also, the wrist-wearable blood pressure measurement device may further include a communication unit configured to transmit and receive data to and from an external device.

According to another aspect of the present disclosure, a method of controlling a blood pressure measurement device includes measuring an angle at which an inner side surface of a wrist of a subject rotates around a vertical reference axis perpendicular to a ground by using an angle sensor included in the blood pressure measurement device, determining that the angle is in a normal state when the measured angle is within an allowable angle range, and determining that the angle is in an abnormal state when the measured angle is not within the allowable angle range, informing the subject that the angle is in the abnormal state when the angle is in the abnormal state, and measuring blood pressure of the subject by using a blood pressure measurement sensor when the angle is in the normal state.

Also, the measuring of the blood pressure may include outputting notification information indicating that the angle is in the normal state.

Also, in the informing the subject, notification information indicating that the rotation state of the wrist is in the normal state or notification information indicating that the rotation state of the wrist is in the abnormal state may be output through vibration notification, voice notification, a display screen output, or an air pressure of a cuff.

### BRIEF DESCRIPTION OF THE DRAWINGS

Embodiments of the inventive concept will be more clearly understood from the following detailed description taken in conjunction with the accompanying drawings in which:

FIG. 1 is a schematic view of a wrist-wearable blood pressure measurement device according to an embodiment of the present disclosure;

FIGS. 2A and 2B are conceptual views of a wrist angle of a wrist-wearable blood pressure measurement device according to an embodiment of the present disclosure;

FIG. 3 is a block diagram illustrating a guide unit of a blood pressure measurement device according to an embodiment of the present disclosure; and

FIG. 4 is a flowchart of a method of controlling a blood pressure measurement device according to an embodiment of the present disclosure.

### DETAILED DESCRIPTION OF THE EMBODIMENTS

Hereinafter, embodiments of the present disclosure will be described in detail such that those skilled in the art to which the present disclosure belongs may easily implement the present disclosure with reference to the accompanying drawings. However, the present disclosure may be implemented in many different forms and is not limited to the embodiments to be described herein. In addition, in order to clearly describe the present disclosure with reference to the drawings, portions irrelevant to the description are omitted, and similar reference numerals are attached to similar portions throughout the specification.

When it is described that a portion is “connected” to another portion throughout the specification, this includes not only a case where the portion is “directly connected” to another portion but also a case where the portion is “indirectly connected” to another portion with another component therebetween.

When it is described that a member is “on” another member throughout the specification, this includes not only a case where a member is in contact with another member, but also a case where there is another member between the two members.

The present disclosure proposes a wrist-wearable blood pressure measurement device 100 that is worn on a subject's wrist to measure the subject's blood pressure.

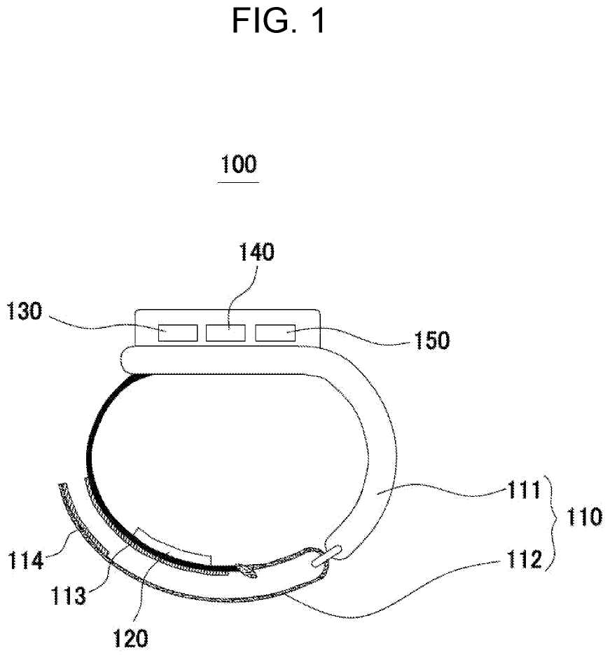

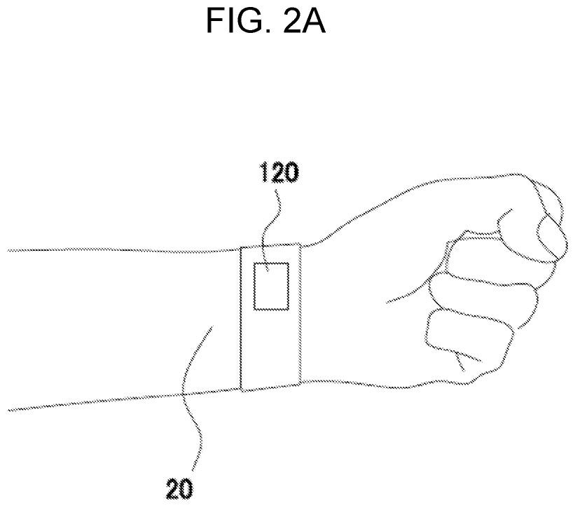

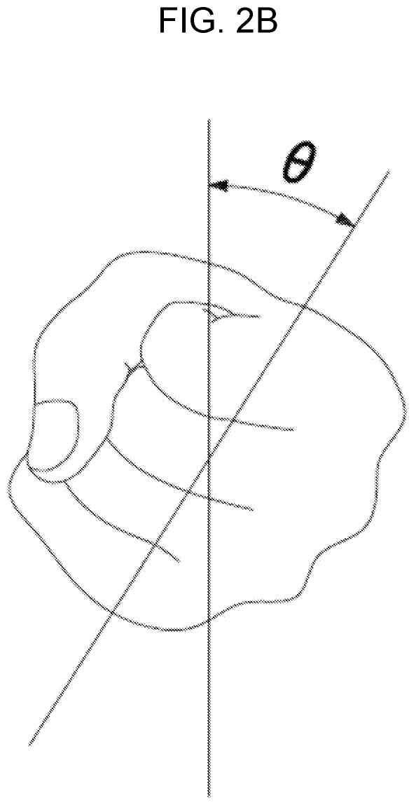

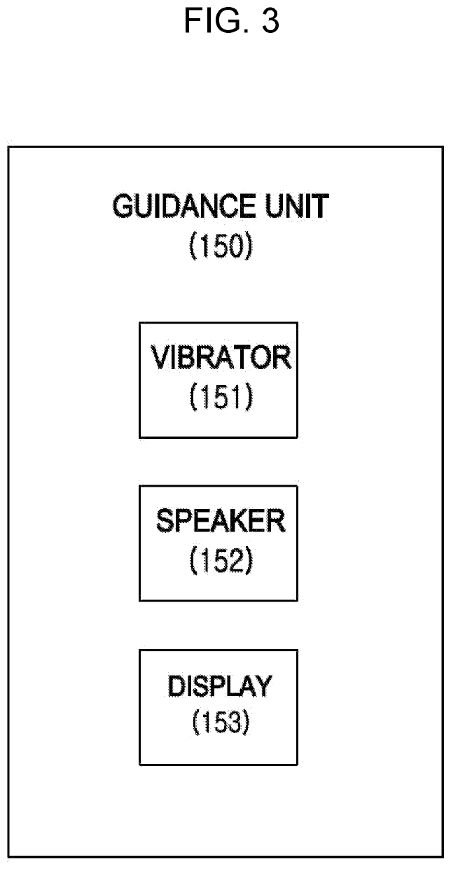

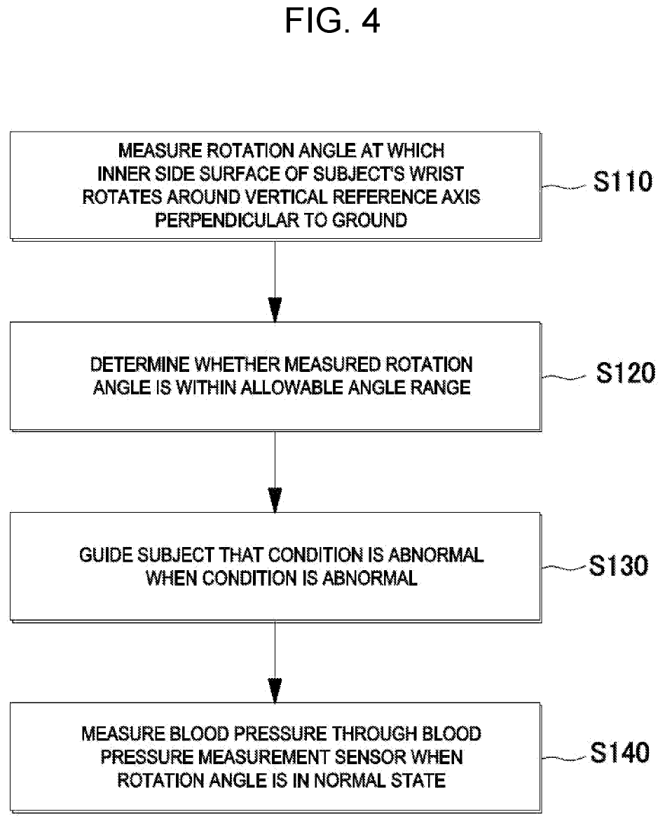

FIG. 1 is a schematic view of a wrist-wearable blood pressure measurement device 100 according to an embodiment of the present disclosure, FIGS. 2A and 2B are conceptual views of a wrist angle of the wrist-wearable blood pressure measurement device 100 according to the embodiment of the present disclosure, FIG. 3 is a block diagram illustrating a guide unit 150 of the blood pressure measurement device 100 according to the embodiment of the present disclosure, and FIG. 4 is a flowchart of a method of controlling the blood pressure measurement device 100, according to an embodiment of the present disclosure.

Hereinafter, the wrist-wearable blood pressure measurement device 100 (hereinafter referred to as “blood pressure measurement device 100”) according to the embodiment of the present disclosure is described with reference to FIGS. 1 to 3.

The blood pressure measurement device 100 may be wearable on a subject's wrist and may include a strap 110 that may wrap the subject's wrist, a blood pressure measurement sensor 120 that may be adjacent to the subject's artery, a blood pressure measurement module 130 that receives data measured by the blood pressure measurement sensor 120, and an angle sensor 140 that detects a rotation state of the subject's wrist.

The strap 110 may include a first strap 111 that wraps one portion of the subject's wrist and a second strap 112 that wraps another portion of the subject's wrist. In addition, the first strap 111 wraps a part of the subject's wrist adjacent to the ulnar artery and is formed of a material having a strength of a preset size or more to prevent the subject's wrist and ulnar artery from being pressurized by the first strap 111, and accordingly, it is possible to prevent the blood flowing through the ulnar artery from being blocked. Also, the second strap 112 may be connected to the first strap 111 to wrap another portion of the wrist and may wrap one portion of the wrist adjacent to the radial artery. For example, the second strap 112 may include a first hook 113 coupled to a middle portion thereof and a second hook 114 in an end portion, and the blood pressure measurement device 100 may be fixed to the wrist by using a Velcro that attaches the first hook 113 to the second hook 114. However, the strap 110 is not limited thereto and may be composed of various commonly known types of straps for fixing a device to the wrist.

The blood pressure measurement sensor 120 may be adjacent to a subject's artery and may measure blood pressure of the subject's artery. For example, the blood pressure measurement sensor 120 may be a pressure sensor but is not limited thereto.

The blood pressure measurement module 130 may receive data measured by the blood pressure measurement sensor 120. Also, the blood pressure measurement module 130 may control the blood pressure measurement sensor 120 to measure blood pressure when blood pressure measurement is needed, such as when receiving blood pressure measurement request information or the blood pressure measurement is automatically set at regular intervals and may display the data measured in this way on a display 153. For example, the blood pressure measurement module 130 may measure blood pressure on an hourly basis, display the measured blood pressure on the display 153, generate an alarm when the blood pressure out of a preset range is measured, and may perform a series of processes in which relevant information may be transmitted to a preset mobile phone, medical facility, and so on by using a communication unit (not illustrated).

The communication unit may communicate with an external device in a set communication format to transmit and receive data, such as measured blood pressure information. Also, the communication unit may receive update information, such as a program related to blood pressure measurement, from an external device and transmit the update information to the blood pressure measurement module 130. By way of example, the communication unit may utilize communication technologies, such as radio frequency identification (RFID), an ultra wide band (UWB), the Bluetooth, and a wireless sensor network that are suitable for data transmission and reception but is not limited thereto.

The angle sensor 140 detects a rotation state of a subject's wrist. In this case, referring to FIG. 2B, the angle sensor 140 measures a rotation angle θ at which an inner side surface 20 of a wrist rotates from a vertical reference axis perpendicular to the ground. In other words, the blood pressure measurement device 100 may measure an appropriate blood pressure of a subject when the inner side surface 20 of a wrist is parallel to the vertical reference axis or when the inner side surface 20 of a wrist is perpendicular to the vertical reference axis to face the sky, and preferably, as illustrated in FIG. 2A, when the inner side surface 20 of the subject's wrist faces an opposite direction to the ground or when the inner side surface 20 faces the sky, the blood pressure measurement device 100 may measure blood pressure in optimal condition. Accordingly, the blood pressure measurement device 100 measures the rotation angle θ at which the inner side surface 20 of a wrist rotates around the vertical reference axis by using the angle sensor 140, and determines whether the rotation angle θ at which the inner side surface 20 of a wrist rotates is within an allowable angle range.

As described above, the blood pressure measurement device 100 determines whether the rotation angle θ at which the inner side surface 20 of a subject's wrist rotates is within an allowable angle range through the data received from the angle sensor 140 and determines whether the rotation angle θ is normal. In other words, the blood pressure measurement module 130 determines that the rotation angle θ is in a normal state when the rotation angle θ of the inner side surface 20 of a wrist received from the angle sensor 140 is within an allowable angle range, and determines that the rotation angle θ is in an abnormal state when the rotation angle θ is out of the allowable angle range. Also, the blood pressure measurement module 130 determines at least one of whether to continue the measurement, whether to provide guidance, and the validity of a measured value based on the rotation angle θ at which the inner side surface 20 of a subject's wrist rotates. In this case, the allowable angle range may be greater than or equal to 0 degrees and less than or equal to 90 degrees. For example, when the inner side surface 20 of a wrist is parallel to the vertical reference axis (that is, a rotation angle is 0 degrees), when the inner side surface 20 of a wrist is perpendicular to the vertical reference axis (that is, the rotation angle is 90 degrees) to face the sky, or when the inner side surface 20 of a wrist is between 0 degrees and 90 degrees with respect to the vertical reference axis, blood pressure may be appropriately measured. However, when the inner side surface 20 of a wrist face the ground, blood pressure may not be appropriately measured. In this case, an alarm may be output to help a user maintain an appropriate wrist rotation state.

In detail, the blood pressure measurement module 130 stops measurement when the rotation angle θ of the inner side surface 20 of a subject's wrist received from the angle sensor 140 is in an abnormal state, and maintain the measurement state when the rotation angle θ is in a normal state. Also, when it is determined that the rotation angle θ at which the inner side surface 20 of a subject's wrist rotates is in an abnormal state, the blood pressure measurement module 130 may guide the subject through the guide unit 150 to adjust the rotation angle θ at which the inner side surface 20 of the wrist rotates. In addition, the blood pressure measurement module 130 sets the blood pressure measurement data measured in an abnormal state as invalid data and sets the blood pressure measurement data measured in a normal state as valid data, thereby calculating a subject's blood pressure based on the valid data.

When it is determined that the rotation angle θ of the inner side surface 20 of the subject's wrist is in a normal state through the blood pressure measurement module 130, the blood pressure measurement device 100 may output notification information informing the subject that the rotation angle θ is in a normal state. Also, when it is determined that the rotation angle θ of the inner side surface 20 of the subject's wrist is in an abnormal state through the blood pressure measurement module 130, the blood pressure measurement device 100 may guide the subject to adjust the inner side surface 20 of the wrist to be parallel to the vertical reference axis or the inner side surface 20 of the wrist to be perpendicular to the vertical reference axis to face the sky. Also, the blood pressure measurement device 100 may further include the guide unit 150 that informs a subject that the subject's wrist angle is in a normal state or an abnormal state.

Referring to FIG. 3, the guide unit 150 may include at least one of a vibrator 151, a speaker 152, and the display 153. Also, the blood pressure measurement device 100 may output notification information indicating that a wrist's rotation state is normal or notification information indicating that the wrist's rotation state is abnormal through a vibration notification, voice notification, display screen output, or cuff air pressure.

The vibrator 151 may inform a subject that a wrist's rotation state is normal or abnormal through vibration. For example, the vibrator 151 may generate one long vibration when the wrist's rotation is normal and may generate several short vibrations when the wrist's rotation is abnormal. In this case, the vibrator 151 may be a vibration motor that vibrates according to an external signal to generate a feeling of tremor, and the vibration motor is the known technology, and accordingly, detailed descriptions thereof are omitted.

The speaker 152 may inform a subject through sound whether a wrist's rotation state is normal or abnormal. In other words, the speaker 152 may output a voice that guides a subject to maintain or change the rotation angle θ of the inner side surface 20 of a wrist for measuring blood pressure based on an angle value measured by the angle sensor 140. Also, when the rotation angle θ at which the inner side surface 20 of a subject's wrist rotates is abnormal based on the angle value measured by the angle sensor 140, the speaker 152 may output a voice guiding a subject to adjust the inner side surface 20 of a wrist to be parallel to the vertical reference axis or the inner side surface 20 of the wrist to be perpendicular to the vertical reference axis to face the sky, and when the rotation angle θ is normal, the speaker 152 may output a voice guiding a subject to reduce movement while measuring blood pressure without changing the rotation angle θ of the wrist. In this case, the output voice may include various voices. such as a recorded voice and a machine voice obtained by modulating a text into voice, and as described above, the voice may include information recommending maintenance or change of the rotation angle θ at which the inner side surface 20 of a subject's wrist rotates. Also, the speaker 152 includes one or more speakers, and may control and select the volume and language type of the output sound through a controller.

The display 153 may inform a subject that a rotation state of a wrist is normal or abnormal. In other words, the display 153 may output information guiding a subject to maintain or change the rotation angle θ of the inner side surface 20 of a subject's wrist for measuring blood pressure based on an angle value measured by the angle sensor 140. Also, when the rotation angle θ at which the inner side surface 20 of a subject's wrist rotates is abnormal based on an angle value measured by the angle sensor 140, the display 153 may output information guiding a subject to adjust the inner side surface 20 of the wrist to be parallel to the vertical reference axis or the inner side surface 20 of the wrist to be perpendicular to the vertical reference axis to face the sky and when the rotation angle θ is normal, the display 153 may output information guiding a subject to reduce movement while measuring blood pressure without changing the rotation angle θ of the wrist. Also, the display 153 may be include various types of display modules, such as a liquid crystal display, a reflective display, and an organic light emitting diode (OLED) display and may display blood pressure or other types of information.

Also, the blood pressure measurement device 100 may inform a subject that the rotation state of a wrist is normal or abnormal through the air pressure of a cuff. The cuff may be adjacent to the artery of a subject's wrist to allow air to flow in from the outside to pressurize the artery of the wrist. In this case, the blood pressure measurement module 130 may calculate a subject's blood pressure by measuring the pressure of the cuff. For example, when a rotation state of a wrist is abnormal, the blood pressure measurement device 100 may periodically repeat supplying air pressure to the cuff or expelling the filled air, and thereby, a subject may check that the rotation of the wrist is abnormal through the cuff but is not limited thereto.

Also, when detecting movement beyond a preset angle range through the angle sensor 140 while measuring blood pressure, the blood pressure measurement device 100 may stop the measurement and may provide guidance to a subject to reduce movement through the vibrator 151, the speaker 152, the display 153, or the air pressure of a cuff.

Hereinafter, a method of controlling the blood pressure measurement device 100, according to an embodiment of the present disclosure, is described with reference to FIG. 4.

In step S110, the angle sensor 140 included in the blood pressure measurement device 100 measures the rotation angle θ at which the inner side surface 20 of a subject's wrist rotates around a vertical reference axis perpendicular to the ground.

In step S120, when the measured rotation angle θ is within an allowable angle range, it is determined that the rotation angle θ is in a normal state, and when the rotation angle θ is not within the allowable angle range, it is determined that the rotation angle θ is in an abnormal state.

In step S130, when a condition is abnormal, a subject is informed that the condition is abnormal. In this case, it can be output that a rotation state of the wrist is abnormal through vibration notification, voice notification, display screen output, or an air pressure of a cuff.

Step S140 includes measuring a subject's blood pressure through the blood pressure measurement sensor 120 when the rotation angle θ is in a normal state. In this case, the blood pressure measurement device 100 may output notification information informing a subject that the rotation angle θ is in a normal state.

According to the embodiments, a wrist-wearable blood pressure measurement device of the present disclosure may use an angle sensor to measure a subject's blood pressure, guide a subject to adjust an inner side surface of the subject's wrist to be parallel to a vertical reference axis or the inner side surface of the wrist to be perpendicular to the vertical reference axis to face the sky, and accurately measure blood pressure by determining the validity of the measured value according to the measurement.

One embodiment of the present disclosure may be implemented in the form of a recording medium including instructions executable by a computer, such as a program module executed by a computer. A computer readable medium may be any available medium that may be accessed by a computer and includes both volatile and nonvolatile media, removable and non-removable media. Also, the computer readable medium may include a computer storage medium. A computer storage medium includes both volatile and nonvolatile media and removable and non-removable media implemented by any method or technology for storing information, such as computer readable instructions, data structures, program modules or other data.

Although the method and systems of the present disclosure are described with reference to specific embodiments, some or all of their components or operations may be implemented by using a computer system having a general-purpose hardware architecture.

The above description of the present disclosure is for illustrative purposes, and those skilled in the art to which the present disclosure belongs will understand that the present disclosure may be easily modified into another specific form without changing the technical idea or essential features of the present disclosure. Therefore, the embodiments described above should be understood as illustrative in all respects and not limiting. For example, each component described as a single type may be implemented in a distributed manner, and likewise, components described as distributed may be implemented in a combined form.

The scope of the present disclosure is indicated by the following claims rather than the detailed description above, and the meaning and scope of the claims and all changes or modifications derived from the equivalent concepts should be interpreted as being included in the scope of the present disclosure.

What is claimed is:

1. A wrist-wearable blood pressure measurement device comprising:
a strap configured to wrap a wrist of a subject;
a blood pressure measurement sensor placed to be adjacent to an artery of the subject;
a blood pressure measurement module configured to receive data measured by the blood pressure measurement sensor; and
an angle sensor configured to detect a rotation state of the wrist of the subject,
wherein the angle sensor measures an angle at which an inner side surface of the wrist rotates around a vertical reference axis perpendicular to a ground, and
the blood pressure measurement module determines that the angle is in a normal state when the angle measured by the angle sensor is within an allowable angle range, and determines the angle is in an abnormal state when the angle measured by the angle sensor is not within the allowable angle range, and determines at least one of whether to continue measurement, whether to provide guidance, and whether a measurement value is valid.

2. The wrist-wearable blood pressure measurement device of claim 1, wherein
the allowable angle range is greater than or equal to 0 degrees and less than or equal to 90 degrees.

3. The wrist-wearable blood pressure measurement device of claim 1, wherein
when the blood pressure measurement module determines that the angle of the wrist of the subject is in a normal state, notification information is output to inform the subject that the angle is in the normal state.

4. The wrist-wearable blood pressure measurement device of claim 1, wherein
when the blood pressure measurement module determines that the angle of the wrist of the subject is in an abnormal state, guidance is provided to the subject to adjust the inner side surface of the wrist to be parallel to the vertical reference axis or the inner side surface of the wrist to be perpendicular to the vertical reference axis to face sky.

5. The wrist-wearable blood pressure measurement device of claim 3, wherein
the wrist-wearable blood pressure measurement device outputs notification information indicating that the rotation state of the wrist is in the normal state or notification information indicating that the rotation state of the wrist is in the abnormal state through vibration notification, voice notification, a display screen output, or an air pressure of a cuff.

6. The wrist-wearable blood pressure measurement device of claim 4, wherein
the wrist-wearable blood pressure measurement device outputs notification information indicating that the rotation state of the wrist is in the normal state or notification information indicating that the rotation state of the wrist is in the abnormal state through vibration notification, voice notification, a display screen output, or an air pressure of a cuff.

7. The wrist-wearable blood pressure measurement device of claim 1, further comprising:
a communication unit configured to transmit and receive data to and from an external device.

8. A method of controlling blood pressure measurement device, the method comprising:
measuring an angle at which an inner side surface of a wrist of a subject rotates around a vertical reference axis perpendicular to a ground by using an angle sensor included in the blood pressure measurement device;
determining that the angle is in a normal state when the measured angle is within an allowable angle range, and determining that the angle is in an abnormal state when the measured angle is not within the allowable angle range;
informing the subject that the angle is in the abnormal state when the angle is in the abnormal state; and
measuring blood pressure of the subject by using a blood pressure measurement sensor when the angle is in the normal state.

9. The method of claim 8, wherein
the allowable angle range is greater than or equal to 0 degrees and less than or equal to 90 degrees.

10. The method of claim 8, wherein
the measuring of the blood pressure includes outputting notification information indicating that the angle is in the normal state.

11. The method of claim 8, wherein
in the informing the subject, notification information indicating that the rotation state of the wrist is in the normal state or notification information indicating that the rotation state of the wrist is in the abnormal state is output through vibration notification, voice notification, a display screen output, or an air pressure of a cuff.
